(19) United States
(12) Patent Application Publication
(10) Pub. No.: US 2008/0154063 A1
(43) Pub. Date: Jun. 26, 2008

(54) Compounds and methods for amino-alkylenediol synthesis

(76) Inventors: Robert O. Cain, Boulder, CO (US); Hendrik Moorlag, Westminister, CO (US); Charles E. Tucker, Superior, CO (US); Jim-Wah Wong, Boulder, CO (US)

Correspondence Address: ROCHE PALO ALTO LLC;Patent Law Department, M/S A2-250, 3431 Hillview Avenue, Palo Alto, CA, 94304, US

(21) Appl. No.: 12/004,190

(22) Filed: Dec. 20, 2007

Related U.S. Application Data
(60) Provisional application No. 60/876,828, filed on Dec. 22, 2006.

(51) Int. Cl. C07C 215/10; C07C 213/00; C07C 215/20

(52) U.S. Cl. 564/443; 564/506

(57) ABSTRACT

A method of making an amino-alkylenediol and intermediate compounds useful in the method is disclosed. The method includes preparing a first intermediate compound comprising an aminoalkylene diol wherein a protecting group is linked to the amino functionality, and optionally, preparing a second intermediate compound comprising a salt of the first intermediate compound.

The first intermediate compound has the structure

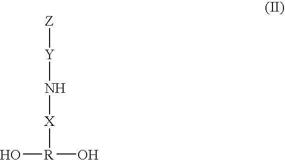

wherein R is a divalent alkylene radical having from 2 to 20 carbon atoms, X and Y are independently a divalent linking moiety or a single bond, and Z is a protecting group.

The second intermediate compound has the structure

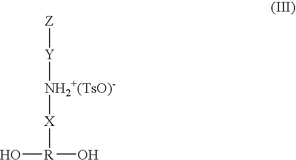

wherein R is a divalent alkylene radical having from 2 to 20 carbon atoms, X and Y are independently a divalent linking moiety or a single bond, TsO⁻ is toluene sulfonate, and Z is a protecting group.

### PRIORITY CLAIM

The present non-provisional patent Application claims benefit from U.S. Provisional Patent Application having Ser. No. 60/876,828, filed on Dec. 22, 2006, by Wong, and titled COMPOUNDS AND METHODS FOR AMINO-ALKYLENEDIOL SYNTHESIS, wherein the entirety of said provisional patent application is incorporated herein by reference.

### FIELD OF THE INVENTION

The present invention relates to compounds and methods useful in the synthesis of amino-alkylenediols. More particularly, the present invention relates to intermediate compounds and the preparation of these intermediate compounds that comprise a protecting group that facilitates the isolation of the amino-alkylenediol in an aqueous environment.

### BACKGROUND

Amino-alkylenediols, such as 3-amino-1,5-pentanediol are introduced as a key fragment in the synthesis of pyridopyrimidines and derivatives thereof such as hydroxyalkyl substituted pyrido-7-pyrimidin-7-ones. The pyridopyrimidines and their derivatives are useful in treating various disorders in a patient and are currently being evaluated as P38(4) MAP Kinase Inhibitors in treatment for rheumatoid arthritis. The preparation of these materials is known. See for example United States Patent Application Publication 2005/0107408 A1 and WO 2002/2064594 A2.

There are various known methods of synthesizing these amino-alkylenediols. However, these methods often involve synthesis in an aqueous system using reactions that require aqueous work up procedures. The hydrophilic nature of the aminodiol moiety makes it difficult and inefficient to manufacture these compounds in high yield. Significant loss in yield often occurs in the final extraction of these products from the aqueous environment. Accordingly, there exists a need for a new method to make these compounds economically and in high yield.

### SUMMARY

The present invention achieves this result. It provides a method and intermediate compounds that facilitates the manufacture of amino-alkylenediols economically and in high yield by allowing isolation of the intermediate compounds in an aqueous environment, and the final product in a non-aqueous environment.

### The amino-alkylenediols have the structure

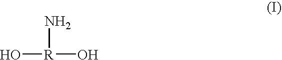

wherein R is an alkylene group having from 2 to 20 carbon atoms. In one embodiment, R in Compound I has the formula —CH₂(CH₂CH(NH₂)CH₂)ₙCH₂₋— wherein n is an integer of from 1 to 6.

In one embodiment, the method of the invention comprises the introduction of an organic “handle” or “handles” such that the intermediate compounds may be extracted from an aqueous environment efficiently. The handle or handles are referred to hereinafter as a protecting group. The protecting group is linked to the amino functionality of the intermediate compounds (Compounds II and III). Compound III comprises a salt of Compound II. The salt may be subsequently reduced to a purified form of the first intermediate compound.

In another aspect of the invention, the first intermediate compound (Compound II) is converted to the amino-alkylenediol (Compound I). A specific embodiment of this method comprises reducing a dialklyl(amino)alkyl enamine (e.g., benzylenaminediester) to a dialkyl(amino)alkyl diester (e.g., benzylaminodiester), subsequently reducing the diester to the first intermediate compound, which may be extracted from an aqueous medium, and then converting the first intermediate directly to the amino-alkylenediol (Compound I) by, for example, removal of the benzyl group(s) via hydrogenation or hydroformylation in a non-aqueous environment.

In yet another aspect of the invention, a purified form of the first intermediate compound is prepared from the second intermediate after which the purified first intermediate compound may be converted to the amino-alkylenediol (Compound I) by, for example, the method(s) set forth in the previous paragraph.

The present invention also provides novel intermediate compounds. One intermediate compound of the invention comprises a laminodiol having a benzyl group linked at the nitrogen. Another intermediate compound of the invention comprises a salt of the benzylamino diol.

The first intermediate compound may be represented by the formula:

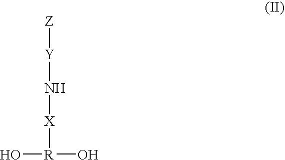

wherein R is as defined above, X and Y are independently a divalent linking moiety or a single bond, and Z is the protecting group.

The second intermediate compound may be represented by the formula:

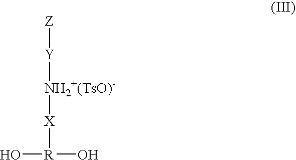

wherein R, X, Y and Z are as described above, and TsO⁻ is p-toluene sulfonate (i.e., CH₃C₆H₄SO₂—).

### DETAILED DESCRIPTION

As used herein, the following terms have the following meanings, unless otherwise indicated:

“Alkyl” means a linear or branched monovalent, saturated hydrocarbon radical having from 1 to 20 carbon atoms that may optionally contain one or more heteroatoms therein.

“Alkylene” means a divalent linear or branched, saturated hydrocarbon radical having from 2 to 20 carbon atoms that may optionally contain one or more heteroatoms.

“Protecting group” means an atom or grouping of atoms that when linked to the amino group reduces the reactivity of the amino group by protecting it from unwanted side reactions. Examples of protecting groups can be found in T. W. Green and P. G. Futs, Protective Groups in Organic Chemistry, (Wiley 2^{nd} ed., 1991) and Harrison and Harrison et al, Compendium of Synthetic Organic Methods, Vols. 1-8 (John Wiley and Sons, 1971-1996). Representative examples of useful protecting groups include formyl, acetyl, trifluoroacetyl, benzyl, benzyloxycarbonyl, tert-butoxycarbonyl, trimethyl silyl, 2-trimethylsilyl-ethanesulfonyl, trityl and substituted trityl, aklyoxycarbonyl, 9-fluoroenylnethyloxycarbonyl, nitro-veratryloxycarbonyl, and combinations thereof.

“Divalent linking moiety” means a moiety that links two atoms or groups together. Examples of divalent linking moieties useful in the invention include an alkylene radical, a cycloalkylene radical, an arylalkylene radical, and combinations thereof.

Turning now to the various embodiments of the invention identified above, the method comprises the preparation of a first intermediate compound (Compound II). This may be accomplished by a variety of methods known in the art. One method for preparing the first intermediate compound is shown in Scheme 1 below.

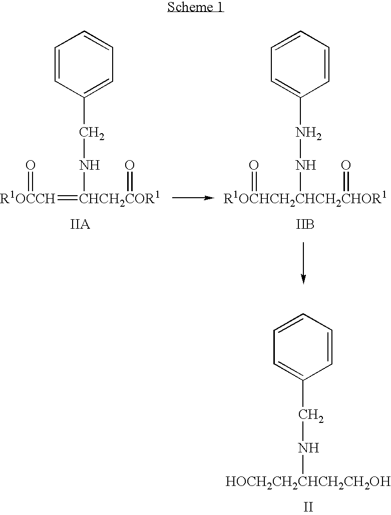

Compound IIB can be prepared by reducing Compound IIA using any of a number of reducing agents. This reduction can be readily carried out in a solvent that is inert under the conditions of the reaction.

Examples of useful reducing agents and acids include sodium boron hydride in acetic acid (NaBH₄/HOAc), sodium boron hydride in methanol (NaBH₄/MeOH), sodium boron hydride in phosphoric acid (NaBH₄/H₃PO₄) and tert-butyl amino borane in sulfuric acid (t-BuNH₂.BH₃ in H₂SO₄, also referred to herein as TBAB), and NaH₂Al(OCH₂CH2)C₂H₅ (also referred to herein as Vitride). Suitable solvents for use in the reaction include toluene, tetrahydrofuran (THF), etc. The reduction can be carried out at a temperature of from about −20° C. to about 120° C. Typically the reduction is carried out at a temperature in the range of from about −20° C. to 40° C.

Reduction of Compound IIB provides the Compound II. This reduction may be carried out with NaBH₄ in an appropriate but optional solvent (e.g., MeOH) in refluxing THF. Less than 3 equivalents of the reducing agent (e.g., NaBH₄) are needed to allow the reaction to go to completion. The reduction can be carried out at a temperature of from about −20° C. to about 120° C. Typically the reduction is carried out at a temperature of from about −10° C. to 30° C.

The method of the invention may comprise the optional step of preparing second intermediate compound (Compound III). This step is useful in removing impurities formed during preparation of the first intermediate (Compound II). An illustrative method for preparing the second intermediate compound is shown in Scheme 2 below.

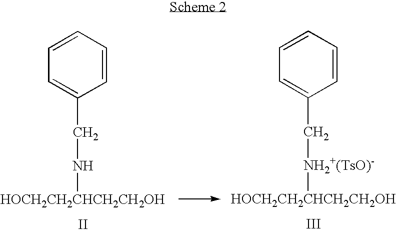

This process comprises forming a tosylate salt of Compound II, crystallizing that salt and isolating that salt by reacting Compound II with an appropriate acid (e.g., para-toluene sulfonic acid), crystallization of the salt in solvent (e.g., THF/CH₂Cl₂) and isolation of the salt.

Conversion of the tosylate salt (Compound III) to a purified form of the first intermediate compound has been found to improve the yield of the desired amino-alkylenediol (Compound I). An illustrative method of preparing a purified version of the first intermediate compound is shown in Scheme 3.

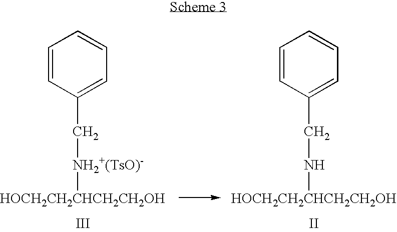

In this method, Compound II is formed from Compound III by adjusting the pH of a solvent solution of Compound II to a pH of from 10-14 and extracting the resulting salt with solvent (e.g., CH₂Cl₂). A recovery of 96% or more can be achieved in this manner.

Compound II may be converted to the desired amino-alkylenediol by a process such as that shown in Scheme 4.

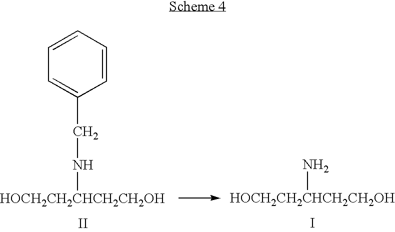

The transformation of Compound II to Compound I may be accomplished by any of a number of processes. For example, Compound II may be dissolved in a solvent or blend of solvents and then charged to a pressure reactor along with a suitable catalyst. The reactor can then be sealed and heated to a desired temperature. The transformation reaction is allowed to proceed, preferably with stirring for a suitable period of time. The transformed product (i.e., Compound I) is then recovered from the reactor.

It is to be appreciated that although the schemes illustrated above indicate exact structures and compositions of the various compounds being prepared, the method of the present invention applies widely to any analogous compounds, given appropriate consideration to protection of the amino functionality employed therein. That is, the amino functionality is shielded from unwanted side reactions during other chemical reactions at other sites on the molecule. The protecting group can then be removed to provide the desired molecule (Compound I).

The preceding discussion has been directed to a multi-step process for forming Compound II. In one version, an enamine (Compound IIA) is converted to a diester (Compound IIB) with subsequent conversion to the diol (Compound II). In another version, an intermediate step is added in which a purified version of Compound II is formed by converting Compound II to a salt (Compound III) and then converting Compound III back to Compound II.

In an alternative embodiment, Compound II may be formed directly from Compound IIA. In this embodiment, the Compound IIA may be reacted with a suitable acid to produce a slurry of the corresponding imine. Thus for example, Compound IIA may be reacted with H₃PO₄ in dimethoxyethane (DME) to produce a slurry of the imine. The imine slurry may then be added to a slurry of NaBH₄ (e.g., 1 eq.) in DME and reacted for a desired time (e.g., 6 hours) with stirring. The reaction mixture may then be added to a second slurry of NaBH₄ (e.q., 3 eq.) in DME, and ethanol and warmed to reflux (e.g., 77° C.) until the reaction is complete. This may take several hours (e.g., 12 hours or more). The reaction mixture may then be cooled to room temperature (e.g., 20° C.) and added slowly to water. A significant amount of hydrogen may be released. When the hydrogen evolution is complete, the pH of the reaction mixture may be adjusted to, e.g., 12-13. The slurry may then be extracted several times with a suitable solvent (e.g., DME) and the resulting organic phases combined to give Compound II. This is typically a pale yellow oil. Yields of greater than 65% can be achieved with this method.

The preparation of Compound II can also be accomplished in a single reaction vessel. An illustrative example of this “one-pot” process is set forth below in Example 1. In this process, at least steps 1A through 1D are carried out in a single reaction vessel.

Additional advantages, features and benefits of the present invention will be apparent to those of skill in the art based upon the following illustrative examples, which are not intended to limit the present invention.

### EXAMPLE 1

3-amino-1,5-pentane diol was prepared according to the following procedure.

Step 1A

Benzylenamine was reduced to benzylamino diester according to the following steps:

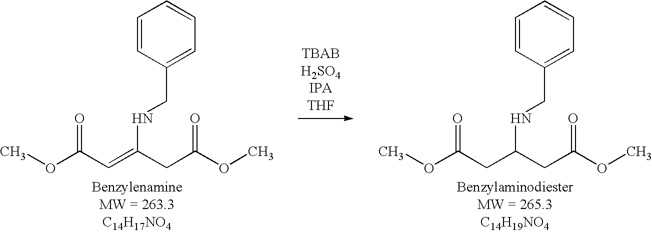

| Material | MW | Mmol | Amount | | Ratio to LR | |
|---|---|---|---|---|---|---|
| Isopropanol (IPA) | | | 16.5 | ml | 1.1 | ml/g |
| Sulfuric acid (conc.) (H₂SO₄) | 98.10 | 113.9 | 11.2 | g | 2 | eq. |
| tert-Butylamine borane complex (TBAB) | 86.97 | 57.0 | 5.0 | g | 1 | eq. |
| Tetrahydrofuran (THF) | | | 82.5 | ml | 3.5 | ml/g |
| Benzylenamine | 263.30 | 57.0 | 15.0 | g | LR | |
| Water | | | 90.0 | ml | 6 | ml/g |
| Ammonium hydroxide (conc.) | | | 11.9 | g | 0.79 | g/g |
| Dichloromethane (DCM) | | | 2 × 52.5 | ml | 3.5 | ml/g |
| Brine (11 wt %) | | | 75.0 | ml | 5 | ml/g |

A vessel is charged with 16.5 ml isopropanol cooled to 0-3° C. Concentrated sulfuric acid (11.2 g) is then slowly added, keeping the addition temperature ≦10° C. Then 5.0 g tert-butylamine borane complex (TBAB) as a solution in 52.5 ml THF is added, keeping the addition temperature ≦10° C. Hydrogen gas is given off through the course of the addition. Then 15.0 g benzylenamine as a solution in 30.0 ml THF is then added, keeping the addition temperature ≦15° C.

After the additions are complete, the batch is warmed to 20-23° C. and stirred for at least 2 hours. A sample is taken to analyze for reaction completion. When the reaction is deemed complete, the batch is added to 90.0 ml water. The THF is then distilled off (150 mmHg/45° C. bath), and 11.9 g concentrated ammonium hydroxide is used to adjust the pH of the batch to 9.5 (±0.5). The batch is then extracted twice with 52.5 ml dichloromethane. The organic layers are combined and extracted once with 75.0 ml 11 wt % brine solution. The organic phase is then stripped (300 to 5 mmHg/45° C. bath) to yield the benzylaminodiester product as a clear colorless oil (97-100% yield based on benzylenamine).

Step 1B

Benzylaminodiol was prepared using the following procedure by reducing the benzylaminodiester.

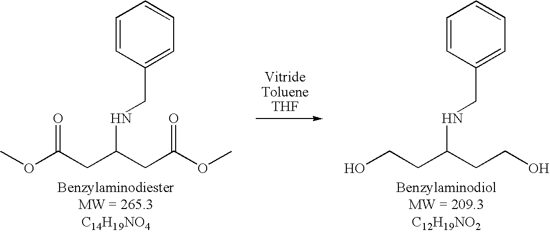

| Material | MW | Mmol | Amount | | Ratio to LR | |
|---|---|---|---|---|---|---|
| Benzylaminodiester | 265.31 | 56.5 | 15.0 | g | LR | |
| Tetrahydrofuran (THF) | | | 90.0 | ml | 6.2 | ml/g |
| Vitride (NaH₂Al(OCH₂CH₂OMe)₂, | 202.17 | 141.3 | 44.0 | g | 2.5 | eq. |
| 65 wt %) | | | | | | |
| 20 wt % Citric acid | | | 225.0 | g | 15 | g/g |
| Water | | | 60.0 | ml | 4 | ml/g |
| 50 wt % NaOH | | | 27.0 | ml | 1.8 | ml/g |
| Dichloromethane (DCM) | | | 2 × 101.0 | ml | 2 × 6.7 | ml/g |

A vessel is charged with 15.0 g benzylaminodiester from step 1A and dissolved in 60.0 ml THF. This is cooled to 0-3° C. Then 44.0 g vitride (65 wt % solution in toluene) is added over approximately 1 hour keeping the addition temperature ≦110° C. The batch is then stirred at 0-10° C. for 30 minutes, and is then sampled for reaction completion. Upon reaction completion, the batch is slowly poured into 225.0 g 20 wt % citric acid (pre-cooled to 0-3° C.), keeping the addition temperature ≦10° C. Hydrogen gas is given off through the course of the addition. The reaction vessel is rinsed into a quench vessel with 30.0 ml water twice and 30.0 ml THF. The pH of the batch is then adjusted to 12.5 (±0.5) with 27.0 ml 50 wt % NaOH. (All solids should dissolve at the elevated pH). The THF is then distilled from the batch (150 mm Hg/45° C. bath). The batch is cooled to 20-23° C., and is then extracted twice with 101.0 ml DCM. The DCM phases are combined and are then concentrated to a total volume of 60 ml.

Step 1C

Benzylaminodiol tosylate was then prepared using the following procedure.

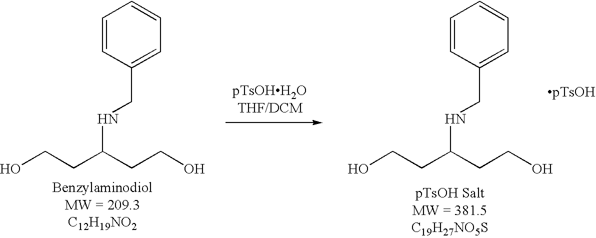

| Material | MW | Mmol | Amount | | Ratio to LR | |
|---|---|---|---|---|---|---|
| Benzylminodiol | 209.29 | 56.4 | 11.8 | g | LR | |
| p-Toluenesulfonic acid | 190.22 | 56.4 | 10.7 | g | 1.0 | eq. |
| hydrate | | | | | | |
| Tetrahydrofuran (THF) | | | 23.6 | ml | 2.0 | ml/g |
| DCM | | | 112.5 | ml | 9.5 | ml/g |
| Benzylaminodiol tosylate | 381.49 | 0.013 | 50 | mg | 0.004 | g/g |

A vessel is charged with 10.7 g para-toluenesulfonic acid hydrate. The hydrate is dissolved in 23.6 ml THF and 65.3 ml dichloromethane and cooled to 5-10° C. The diol solution from Step 1B is slowly added to the pTsOH solution, keeping the addition temperature ≦10° C. When approximately 60% of the diol solution is added, 50 mg seed crystals of benzylaminodiol tosylate are added, and the batch is allowed to crystallize for 15 minutes. Addition of the diol solution is resumed, keeping the temperature ≦10° C. The addition line is rinsed with a small amount of DCM. After the addition is complete, the batch is stirred for at least 2 hours at 5-10° C. The batch is then filtered, and the cake is rinsed 4 times with 11.8 ml dichloromethane. The salt is dried (10 mmHg/40° C./4 h) to yield the benzylaminodiol tosylate as a bright white solid (88-90% yield from the diester).

Step 1D

Purified benzylaminodiol was prepared according to the following procedure.

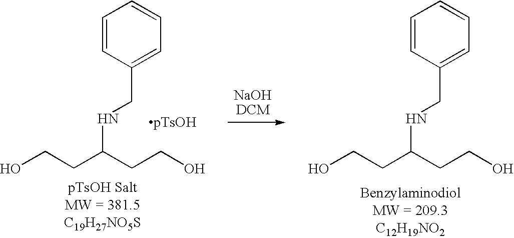

| Material | MW | Mmol | Amount | | Ratio to LR | |
|---|---|---|---|---|---|---|
| Benzylaminodiol tosylate | 381.49 | 50.3 | 19.2 | g | LR | |
| DCM | | | 132.9 | ml | 7.23 | ml/g |
| 1N NaOH | | | 62.9 | ml | 1.25 | eq. |
| Water | | | 55.4 | ml | 1.1 | L/mol |

Benzylaminodiol tosylate from step 1C (19.2 g) is slurried in 132.9 ml dichloromethane and 55.4 ml water in a suitable vessel. Then 62.9 ml 1N NaOH is added to adjust the pH of the aqueous phase to 12.5 (±0.5). This two-phase mixture is stirred for 15 minutes and allowed to settle. The bottom organic phase is separated to a separate flask. The organic phase is heated to reflux, and the condensate is cycled through the aqueous phase to achieve a sort of continuous extraction. Samples are taken of the aqueous phase. The extraction is continued until analysis shows no diol remaining in the aqueous phase. The organic phase is then stripped (300 to 5 mmHg/45° C. bath) to yield the benzylaminodiol a clear colorless oil (92-94% yield based on the tosylate salt).

Step 1E

3-amino-1,5-pentane diol was prepared according to the following procedure.

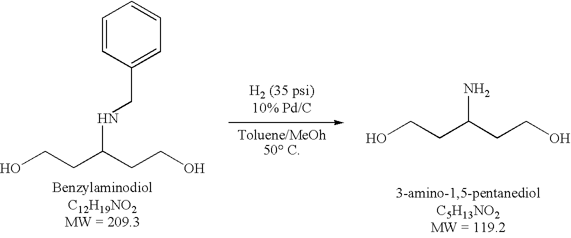

| | | | | |
|---|---|---|---|---|
| Material | MW | Mmol | Amount | Ratio to LR |

| | | | | | |
|---|---|---|---|---|---|
| Benzylaminodiol | 209.29 | 239 | 50.0 | g | LR |
| 10 wt % Pearlman's Catalyst (50% wet) | | | 3.4 | g | |
| Methanol | | | 140 | mL | |
| Toluene | | | 140 | mL | |
| Celite | | | 5 | g | |
| Methanol | | | 50 | mL | |
| 10 wt % Sodium Sulfite | | | 15 | mL | |

A pressure reactor is charged with 50% wet Pearlman's Catalyst. The benzylaminodiol from step 1D (50.0 g) is dissolved in methanol (140 mL) and toluene (140 mL), which is then charged to the reactor. The reactor is sealed, purged (5×35 psi H₂), pressurized with hydrogen (35 psi), and warmed to 50° C. The batch is stirred under pressure at 50° C. for 4 hours. After venting, the batch was filtered through a Celite pad (5 g) and the catalyst pad is rinsed with methanol (2×25 mL). The solvents are then removed under reduced pressure (150 to 5 mm Hg, 45° C. bath) to give 3-amino-1,5-pentanediol product as clear colorless oil (quantitative yield).

### EXAMPLE 2

3-amino-1,5-pentane diol was prepared according to the following procedure.

Step 2A

Benzylamino diester was prepared as described in Step 1A of Example 1.

Step 2B

Benzylaminodiester was reduced to benzylaminodiol according to the following procedure.

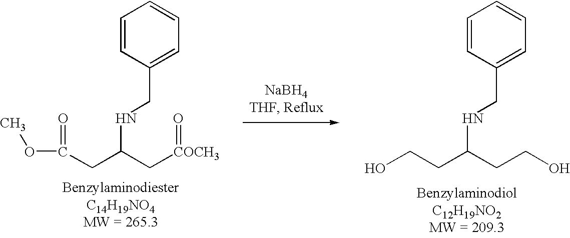

| Material | MW | Mmol | Amount | | Ratio to LR | |
|---|---|---|---|---|---|---|
| Benzylaminodiester | 265.3 | 379.6 | 100.7 | g | LR | |
| THF | | | 534 | ml | 5.3 | ml/g |
| Sodium borohydride (NaBH₄) | 37.83 | 1518.2 | 57.4 | g | 4 | eq. |
| Methanol | | | 71 | ml | 0.71 | ml/g |
| Water | | | 1510 | ml | 15 | ml/g |
| 3N HCl | | | 297 | ml | 2.95 | ml/g |
| DCM | | | 3217 | ml | 31.95 | ml/g |
| 50% NaOH | | | 212 | ml | 2.11 | ml/g |

In a 2 L 4-necked reactor equipped with a large nitrogen inlet, a thermocouple, a reflux condenser, and a mechanical agitator, sodium borohydride (57.4 g) is charged and slurried in THF (8 mL). In a separate flask, the benzylaminodiester (100.7 g) is dissolved in THF (453 mL), and the resulting solution is added directly to the borohydride slurry. Methanol (72 mL) is then added with vigorous stirring. Hydrogen gas is evolved during and after the addition, so the vessel is adequately swept with nitrogen. The batch is then warmed to reflux (56° C. internal temperature, 60° C. bath) and stirred for at least 12 hours, and tested for reaction completion.

Upon reaction completion, the batch is cooled to 0-5° C. and added to water (1510 mL; no hydrogen evolution). The batch is then stripped under reduced pressure (200 mm Hg/45° C. bath) to remove the organic solvents. The pH of the batch is then adjusted to 2-3 with 3N HCl. Acidification of the batch causes hydrogen gas to be evolved. The vessel is well vented, and the acid is added slowly to avoid excess frothing. The addition is exothermic, and the batch is kept ≦10° C. through the course of the acidification. When the desired pH range is reached, the batch is warmed to 20° C. and dichloromethane (297 mL) is added, the batch is stirred for 15 minutes, settled for 30 minutes, and phase separated. The organic phase is discarded. The product-containing aqueous (top) phase is then cooled and made basic (pH to 11-12) by the addition of 50% sodium hydroxide. This addition is also exothermic, and cooling is required to keep the batch temperature ≦110° C. The batch is then warmed to 20° C., and is extracted five times with a dichloromethane (584 mL each). The organic layers are combined and concentrated to benzylaminodiester as a clear colorless to clear yellow oil.

Steps 2C and 2D

Purified benzylamino diol was then prepared as described in Steps 1C and 1D of Example 1

Step 2E

3-amino-1,5-pentane diol was prepared according to the following procedure.

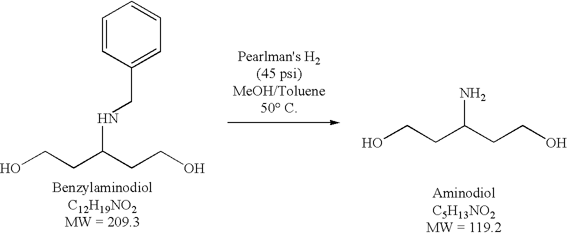

| | | Th. | | | | |
|---|---|---|---|---|---|---|
| Material | MW | Mmol | Th. Measure | | Ratio to LR | |
| Benzylaminodiol | 209.29 | 238.9 | 50.0 | g | LR | |
| Pearlman's Catalyst | | | 3.4 | g | 0.067 | g/g |
| Methanol | | | 140.0 | ml | 2.8 | ml/g |
| Toluene | | | 140.0 | ml | 2.8 | ml/g |
| Celite | | | 5.0 | g | 0.1 | g/g |
| Methanol | | | 50.0 | ml | 1 | ml/g |

Operations:

The Pearlman's catalyst is charged to a pressure reactor. The benzylaminodiol, as a solution in toluene, is then charged into the reactor. Methanol is charged to the reactor. The reactor is then sealed and purged 3× with hydrogen. The reactor is warmed to 50° C. with stirring and held at temperature for 2 hours. The reactor is then vented and a sample is taken to measure for reaction completion. Once the reaction is complete, the batch is cooled and filtered through a Celite. The Celite is rinsed with methanol. The catalyst is deactivated with a 10 wt % sodium sulfite rinse. The batch is then vacuum distilled down to the 3-amino-1,5-pentane diol as an oil.

### EXAMPLE 3

Benzyl enamine to benzyl aminodiol without intermediate isolation.

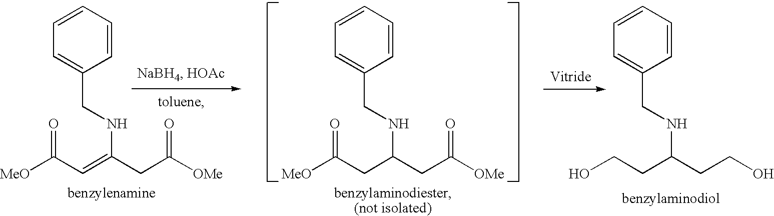

| | | Th. | | | | |
|---|---|---|---|---|---|---|
| Material | MW | Mmol | Th. Measure | | Ratio to FL | |
| Benzylenamine | 263.31 | 76.0 | 20.0 | g | LR | |
| Toluene | | | 80.0 | ml | 4 | ml/g |
| Acetic acid (HOAc) | 80.05 | 342.2 | 19.6 | ml | 4.505 | eq. |
| Sodium borohydride | 37.83 | 132.9 | 5.0 | g | 1.75 | eq. |
| Toluene | | | 100.0 | ml | 5 | ml/g |
| Vitride (70%) | | | 73.0 | ml | 3.65 | g/g |
| HCl (30%) | | | As specified | | | |
| Citric acid (10%) | | | As specified | | | |
| Sodium Hydroxide (20%) | | | As specified | | | |
| Water | | | As specified | | | |
| Dichloromethane | | | 700.0 | ml | 35 | ml/g |
| Toluene | | | 400.0 | ml | 20 | ml/g |

To a stirred slurry of NaBH₄ in 100 mL of toluene cooled to −10° C. in a 1000 mL 3-necked round bottom flask equipped with overhead mechanical stirrer and a thermometer was added a solution of benzylenamine and HOAc in 80 mL of toluene in 20 min. The addition funnel was rinsed with 20 mL of toluene. The batch was allowed to stir and warm up to 15-18° C. slowly overnight. The 16 h HPLC sample showed the enamine had been consumed. The mixture was cooled back to −10° C. and Vitride was added via an addition funnel slowly in 1 hour. The batch was kept below 0° C. during the addition. After the addition, the light yellow milky mixture was stirred at a temperature of between −5 to 0° C. HPLC after 30 min. showed 0.7% diester left. The slurry was stirred for a total of 2 hours. The batch was quenched by adding slowly to a mixed solution of 80 mL of 30% aqueous HCl and 80 mL of 10% aqueous citric acid cooled at 5° C. in 20 min. The temperature of the batch was not allowed to exceed 20° C. The pH at this time was about 1-2. The addition funnel was rinsed with a mixture of 20 mL of 30% aqueous HCl and 20 mL of 10% aqueous citric acid. After warming up to room temperature, the layers were separated. The organic layer was extracted with 100 mL of 10% aqueous citric acid. The combined aqueous portion was then cooled to 0-5° C., and basicified to pH 12.5 with 245 mL of 20% aqueous NaOH. Another 50 mL of water was used for rinsing. The cloudy mixture was then extracted with 7×100 mL of CH₂Cl₂. During the extraction, the aqueous layer was cloudy but the organic layer remained clear and easily separated. The combined organic portion was stripped to an oil. Then 2×200 mL of toluene was added and the solution was stripped to a yellow oil. Toluene was then added to the residue to give 46.2 g of a yellow benzylaminodiol solution.

### EXAMPLE 4

Benzylamindiol of step 2B may be converted to the aminodiol without the use catalytic hydrogenation. In this case, hydrogen transfer is used to remove the benzyl protecting group. Thus, 3-N-benzylpentane-1,5-diol (104.5 mg, 0.5 mmol, 1 equiv) is dissolved in a slurry of 10% Pd/C (100 mg/56% wet) in anhydrous methanol (7 mL). To this is added the proton source, ammonium formate (315 mg, 5 mmol, 10 equiv) and the mixture is refluxed for 40 minutes. The hot solution is filtered through celite and washed with methylene chloride (15 mL). Evaporation gives a clear colorless oil (67 mg). Carbon 13 NMR shows the expected three carbon resonances and proton NMR also confirms the material as the desired product. Gas chromatography after derivatization shows the presence of both the disiloxy and trisiloxy derivatives as separate peaks.

Other embodiments of this invention will be apparent to those skilled in the art upon consideration of this specification or from the practice of the invention disclosed herein. Various omissions, modifications, and changes to the principles and embodiments described herein may be made by one skilled in the art without departing from the true scope and spirit of the invention which is indicated by the following claims.

1. A method for the synthesis of an aminoalkyl diol comprising the step of preparing a first intermediate compound comprising an aminoalkylene diol wherein the amino functionality comprises a protecting group.

2. The method of claim 1 comprising the further steps of (i) preparing a second intermediate compound comprising a salt of the first intermediate compound and (ii) preparing a purified aminoalkylene diol from the second intermediate.

3. The method of claim 1 wherein the step of preparing the first intermediate compound comprises reducing a dialkyl(amino)alkyl enamine to a dialkyl(amino) alkyl diester and reducing the dialkyl(amino) alkyl diester to the first intermediate compound.

4. The method of claim 1 wherein the first intermediate compound comprises the formula 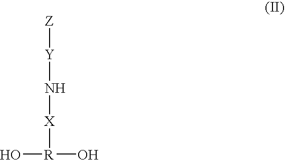
wherein R is a divalent alkylene radical, X and Y are independently a divalent linking moiety or a single bond, and Z is the protecting group.

5. The method of claim 4 wherein R comprises five carbon atoms, X is a single bond, and Y is a divalent linking moiety.

6. The method of claim 1 wherein Z comprises an atom or grouping of atoms that when attached to the amino group reduces the reactivity of the amino group.

7. The method of claim 6 wherein Z is selected from formyl, acetyl, trifluoroacetyl, benzyl, benzyloxycarbonyl, tert-butoxycarbonyl, trimethyl silyl, 2-trimethylsilyl-ethanesulfonyl, trityl and substituted trityl, aklyoxycarbonyl, 9-fluoroenylmethyloxycarbonyl, nitro-veratryloxycarbonyl, and combinations thereof.

8. The method of claim 2 wherein the second intermediate compound comprises the formula 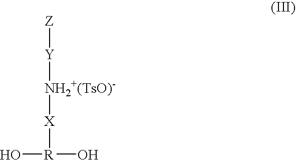
wherein R is a divalent alkylene radical having from 2 to 20 carbon atoms, X and Y are independently a divalent linking moiety or a single bond, TsO⁻ is toluene sulfonate, and Z is a protecting group.

9. The method of claim 1 wherein the aminoalkyl diol has the formula 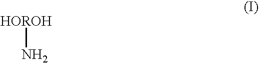
wherein R is a divalent alkylene radical having from 2 to 20 carbon atoms.

10. The method of claim 1 wherein the first intermediate compound is derived from a benzyl enamine of the formula 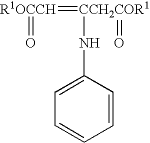
wherein R¹ is an alkyl group having from 1 to 20 carbon atoms.

11. A method for the synthesis of HOCH₂CH₂CH(NH)CH₂CH₂OH comprising
preparing a first intermediate compound having the structure 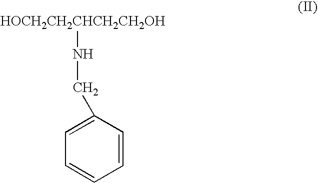
preparing a second intermediate from the first intermediate, the second intermediate comprising the formula 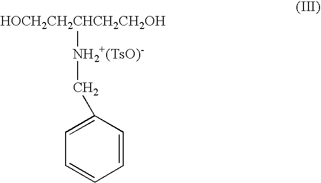
wherein TsO⁻ is p-toluene sulfonate.

12. A compound comprising:
a hydroxyl terminated divalent alkylene radical having from 2 to 20 carbon atoms;
an amino group linked to one of the carbon atoms in the backbone; and
a protecting group linked to the amino group.

13. The compound of claim 12 wherein the amino group is linked to the third carbon atom in the alkylene radical.

14. The compound of claim 12 wherein the protecting group comprises an atom or grouping of atoms that when linked to the amino group reduces the reactivity of the amino group.

15. The compound of claim 14 wherein the protecting group is selected from formyl, acetyl, trifluoroacetyl, benzyl, benzyloxycarbonyl, tert-butoxycarbonyl, trimethyl silyl, 2-trimethylsilyl-ethanesulfonyl, trityl and substituted trityl, aklyoxycarbonyl, 9-fluoroenylmethyloxycarbonyl, nitro-veratryloxycarbonyl, and combinations thereof.

16. The compound of claim 12 comprising the formula the formula:

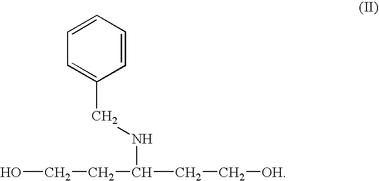

17. A compound comprising:
a hydroxyl terminated divalent alkylene radical having from 2 to 20 carbon atoms;
an amino tosylate group linked to one of the carbon atoms in the divalent alkylene radical; and
a protecting group linked to the amino tosylate group.

18. The compound of claim 17 wherein the amino group is attached to the third carbon atom in the divalent alkylene radical.

19. The compound of claim 17 wherein the protecting group comprises an atom or grouping of atoms that when attached to the amino group reduces the reactivity of the amino group.

20. The compound of claim 17 comprising the formula: 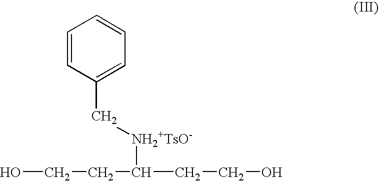
where TsO⁻ represents toluene sulfonate.
